(19) United States
(12) Patent Application Publication
(10) Pub. No.: US 2026/0306100 A1
(43) Pub. Date: Oct. 8, 2026

(54) AN APPARATUS FOR ANASTOMOSING A CUT VESSEL

(71) Applicant: VANDERBILT UNIVERSITY, Nashville, TN (US)

(72) Inventor: Kent K. HIGDON, Nashville, TN (US)

(21) Appl. No.: 18/996,673

(22) PCT Filed: Jul. 19, 2023
(86) PCT No.: PCT/US2023/028164

§ 371 Date: Jan. 17, 2025

Related U.S. Application Data
(60) Provisional application No. 63/390,549, filed on Jul. 19, 2022.

(51) Int. Cl. A61B 17/11

(52) U.S. Cl. CPC A61B 17/11; A61B 2017/1107; A61B 2017/1125; A61B 2017/1132

(57) ABSTRACT

An apparatus for anastomosing a cut vessel includes a first ring. The first ring has an inner surface defining an inner lumen configured to selectively receive a first vessel end of the cut vessel. A second ring has an inner surface that defines an inner lumen configured to selectively receive a second vessel end of the cut vessel. A closure mechanism has a first engagement portion joined to the first ring and a second engagement portion joined to the second ring. A biasing member of the closure mechanism is configured to urge the first and second rings toward one another to anastomose the cut vessel after the first and second vessel ends have been received in the first and second rings.

### RELATED APPLICATION

This application is a U. S National Stage under 35 USC 371 of PCT Application Serial No. PCT/US2023/028164, filed on 19 Jul. 2023, which claims priority from U.S. Provisional Application No. 63/390,549, filed 19 Jul. 2022, the subject matter of which is incorporated herein by reference in its entirety.

### TECHNICAL FIELD

This disclosure relates to an apparatus and method for anastomosing a cut vessel.

### BACKGROUND

Microsurgical vessel connections called anastomoses may be performed with either classic suture techniques—which can take as long as two hours or more per vessel, due to complexity of multiple tiny microsutures being placed in the vessel—or, in the case of veins, with a vein coupler device. A vein coupler device may include two separate plastic rings. Each plastic ring has longitudinally extending metallic spikes. During a vein anastomosis, a medical professional rolls the edges of a cut end of a vein onto the spikes. Using a forceps or another device, the edges of the cut end of the vein are tamped down onto the spikes. The plastic rings are then joined to a delivery device that compresses the spikes of one of the plastic rings into receiving holes on the other of the plastic rings.

Arteries can be much less distensible, less compliant, and thicker than veins. Arteries also have different vessel wall characteristics than veins. Therefore, vein coupler devices, being designed for coupling veins, may be inadequate when used for arterial anastomoses.

### SUMMARY

In an aspect, alone or in combination with any other aspect, an apparatus for anastomosing a cut vessel comprises a first ring. The first ring has an inner surface defining an inner lumen configured to selectively receive a first vessel end of the cut vessel. A second ring has an inner surface that defines an inner lumen configured to selectively receive a second vessel end of the cut vessel. A closure mechanism has a first engagement portion joined to the first ring and a second engagement portion joined to the second ring. A biasing member of the closure mechanism is configured to urge the first and second rings toward one another to anastomose the cut vessel after the first and second vessel ends have been received in the first and second rings.

In an aspect, alone or in combination with any other aspect, a method anastomosing a cut vessel comprises providing the apparatus. The closure mechanism is manipulated into an opened condition. With the closure mechanism in the opened condition, the first ring is positioned over a first vessel end of the cut vessel so that the first vessel end is at least partially within the inner lumen and surrounded by the inner surface of the first ring. With the closure mechanism in the opened condition, the second ring is positioned over a second vessel end of the cut vessel so that the second vessel end is at least partially within the inner lumen and surrounded by the inner surface of the second ring. With the first and second rings positioned over the first and second vessels ends, the closure mechanism is manipulated to a closed condition to urge the first and second rings toward one another and the first and second vessel ends into engagement with one another to anastomose the cut vessel.

In an aspect, alone or in combination with any other aspect, a system for anastomosing a cut vessel comprises the apparatus and an applicator for selectively engaging first and second actuating portions of the closure mechanism and selectively manipulating the first and second actuating portions to move the closure mechanism between opened closed conditions.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a side view of an anastomotic coupler device according to an aspect of the present invention, including the device in a first condition;

FIG. 2 is a top view of the aspect of FIG. 1, including the device in the first condition;

FIG. 3 is a top view of the aspect of FIG. 1, including the device in a second condition;

FIG. 4 is a perspective rear view of a portion of the aspect of FIG. 1;

FIG. 5 is a perspective view of an example configuration of an element of the aspect of FIG. 1;

FIG. 6 is perspective view of an example configuration of an element of the aspect of FIG. 1;

FIG. 7 is a perspective view of an applicator according to an aspect of the present invention; and

FIG. 8 is a cross-sectional view of an example configuration of the device of the aspect of FIG. 1, including the device in an example use environment.

### DETAILED DESCRIPTION

Unless defined otherwise, all technical and scientific terms used herein have the same meaning as is commonly understood by one of ordinary skill in the art to which the present disclosure pertains.

As used herein, the term “user” can be used interchangeably to refer to an individual who prepares for, assists with, and/or performs a procedure.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that when an element is referred to as being “on,” “attached” to, “coupled” with, “contacting,” etc., another element, it can be directly on, attached to, coupled with or contacting the other element or intervening elements may also be present. In contrast, when an element is referred to as being, for example, “directly contacting” another element, there are no intervening elements present.

Spatially relative terms, such as “over” and the like, may be used herein for ease of description to describe one element or feature's relationship to another element(s) or feature(s) as illustrated in the Figures. It will be understood that the spatially relative terms can encompass different orientations of a device in use or operation, in addition to the orientation depicted in the Figures. For example, if a device in the Figures is inverted, elements described as “over” other elements or features would then be oriented “under” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

The invention comprises, consists of, or consists essentially of the following features, in any combination.

A device according to aspects of the present disclosure solves the problem of prior art anastomotic coupler devices, which may be inadequate when used for arterial anastomosis due to their design being based only upon on the characteristics of veins. The wall of an artery is not like a vein wall. Veins are thinner, more distensible, and more easily everted over an edge of an anastomotic coupler device than are arteries. Everting arteries and veins helps to achieve intima-to-intima coaptation during an anastomosis, which helps preclude the unwanted creation of a thrombosis (clotting failure of the vessel connection) during the anastomosis. A device according to the aspects of the prevent disclosure overcomes the issues of the prior art anastomotic coupler devices by being designed for arterial use.

The device disclosed herein also cuts down on the cost of an arterial anastomosis by reducing the time it takes to complete the anastomosis. Because prior art anastomotic coupler devices are inadequate when used for arterial anastomoses, medical professionals are left with using multiple tiny microsutures that can take 30-60 minutes per anastomosis on average and can take as long as 120 minutes or more in complex cases. Use of the device of the present disclosure for an arterial anastomosis may cut the average anastomosis time from 30-60 minutes down to around 5 minutes. Thus, the device of the present invention may reduce the cost of surgery by reducing operating room time for arterial anastomoses.

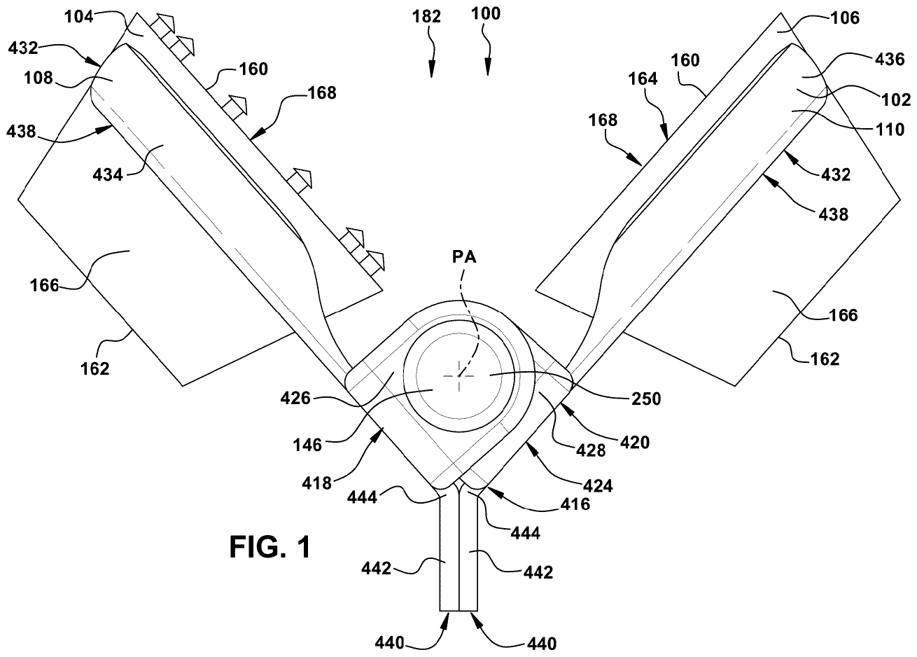

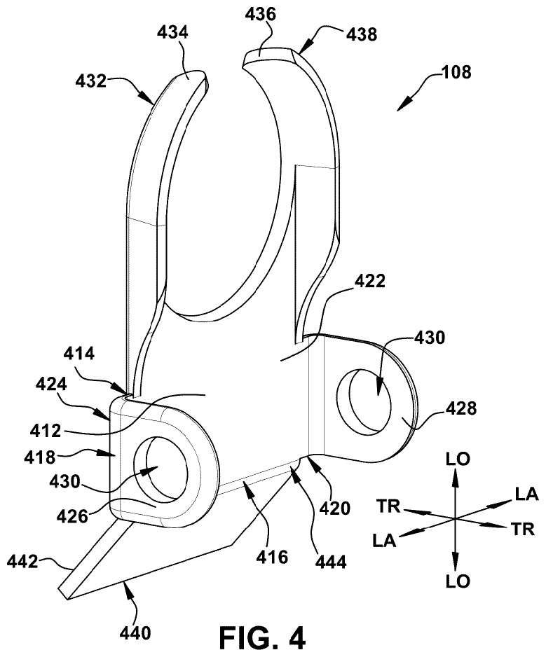

FIGS. 1-3 depict an example anastomotic coupler device 100 designed in accordance with the teachings of the present disclosure. The device 100 includes a closure mechanism 102 having first and second rings 104, 106 selectively joined thereto. The closure mechanism 102 includes first and second wings 108, 110. As shown in FIG. 4, the first wing 108 includes a base plate 412 having opposing first and second longitudinal ends 414, 416 and opposing first and second lateral ends 418, 420 extending longitudinally between the first and second longitudinal ends 414, 416. The term “longitudinal” is used herein to indicate a substantially vertical direction, in the orientation of FIG. 4, and is indicated at “LO” in FIG. 4. The term “lateral” is used herein to indicate a direction substantially perpendicular to the “longitudinal” direction, and is indicated at “LA” in FIG. 4. The base plate 412 also includes opposing first and second transverse surfaces 422, 424. The term “transverse” is used herein to indicate a direction substantially perpendicular to both the “longitudinal” and “lateral” directions, and is indicated at “TR” in FIG. 4.

First and second connection projections 426, 428 extend from the second transverse surface 424, though the first and second connection projections 426, 428 may extend from any other portion of the base plate 412. The first and second connection projections 426, 428 are also spaced laterally from one another on the second transverse surface 424. For example, the first connection projection 426 may be located adjacent the first lateral end 418, while the second connection projection 428 may be located adjacent the second lateral end 420. Each of the first and second connection projections 426, 428 may include an opening 430 extending laterally therethrough. Although the first wing 108 is shown as having two connection projections 426, 428, the first wing 108 may have any desired number of connection projections.

An engagement portion 432 is shown in FIG. 4 as being connected to (e.g., extending from) the first longitudinal end 414 of the base plate 412, though the engagement portion 432 may be connected to any other portion of the base plate 412. The engagement portion 432 is configured to selectively receive the first ring 104 and selectively maintain the first ring 104 engaged thereto. In the example configuration shown in FIG. 4, the engagement portion 432 includes first and second arcuate engagement arms 434, 436 that form a split ring 438. As shown in FIGS. 1-3, the split ring 438 selectively extends around the first ring 104. The split ring 438 thus may be configured such that the first ring 104 is selectively maintained in/engaged to the split ring 438 via a press-fit or frictional engagement.

Returning to FIG. 4, an actuating portion 440 is connected to (e.g., extends from) the second longitudinal end 416 of the base plate 412, though the actuating portion 440 may be connected to any other portion of the base plate 412. The actuating portion 440 may include a substantially flat gripping surface 442 for a user to easily engage and manipulate the first wing 108 with their fingers and/or with a tool during use of the device 100. The gripping surface 442 may be connected to the second longitudinal end 416 via a bent portion 444. The actuating portion 440 thus may be formed via bending a portion of the first wing 108 in transverse direction away from the first transverse surface 422, though the actuating portion 440 may be formed in any other desired manner. Although the bent portion 444 is shown as having a substantially smooth bend forming a curved surface, the bent portion 444 may have a sharper bend forming a more acutely angled surface.

At least one of the base plate 412, the first connection projection 426, the second connection projection 428, the engagement portion 432, and the actuating portion 440 may be integrally formed as a single monolithic piece with at least one other of the base plate 412, the first connection projection 426, the second connection projection 428, the engagement portion 432, and the actuating portion 440, or may be formed separately from and subsequently connected to at least one other of the base plate 412, the first connection projection 426, the second connection projection 428, the engagement portion 432, and the actuating portion 440.

As shown in FIGS. 1-3, the structure of the second wing 110 may substantially mirror the structure of the first wing 108. Therefore, the second wing 110 includes a base plate 412, first and second connection projections 426, 428, an engagement portion 432, and an actuating portion 440. However, the lateral distance between the first and second connection projections 426, 428 of the second wing 110 may be at least slightly different than that of the first and second connection projections 426, 428 of the first wing 108 so that the openings 430 may all be laterally aligned on a single laterally extending pivot axis PA when the first and second wings 108, 110 are joined together.

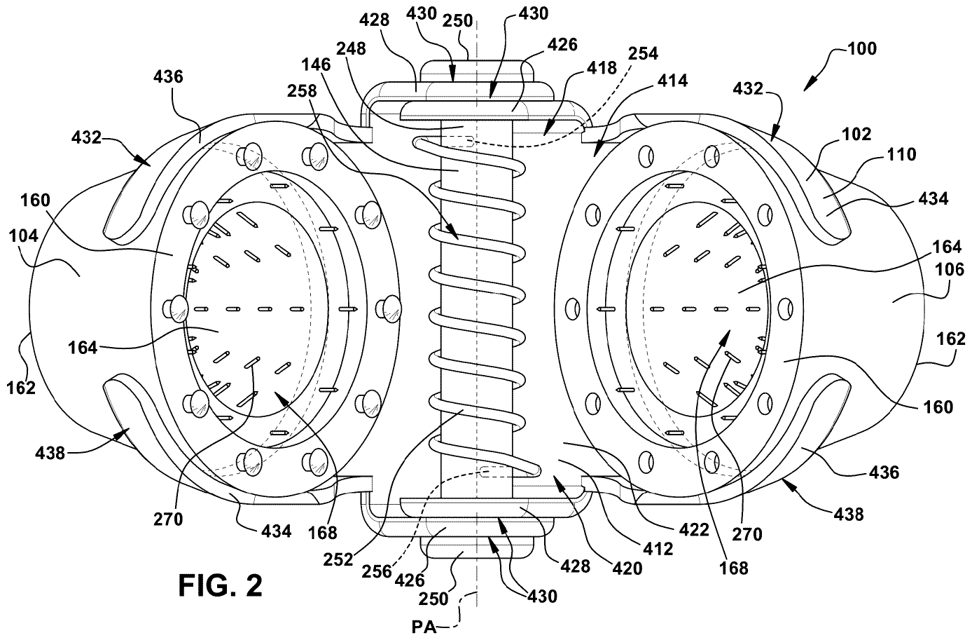

As shown in FIGS. 1-3, the first and second wings 108, 110 are joined together and maintained joined together via a fastener 146. As shown in FIG. 2, the fastener 146 has a shaft 248 received in the aligned openings 430. At opposite ends of the shaft 248, the fastener has enlargements 250 that are too large to pass through the aligned openings 430. Each of the enlargements 250 may be permanently fixed to the shaft 248, as by being formed integrally as a single monolithic piece with the shaft 248, or may be removable from the shaft 248, as by threadably engaged with or otherwise connected to the shaft 248. The fastener 146 may be a rivet, if it is desired to join the first wing 108 to the second wing 110 in a permanent or nonremovable manner, or may be a bolt with a removably attached nut, if it is desired to selectively join the first wing 108 to the second wing 110, for example, for replacement of at least one of the first and second wings 108, 110.

The shaft 248 of the fastener 148 provides a laterally oriented axle or shaft for pivotal movement of the first and second wings 108, 110 relative to one another. Thus, the second wing 110 is joined to the first wing 108 such that the first and second wings 108, 110 are pivotable about the pivot axis PA, which is defined by the shaft 248, relative to each other.

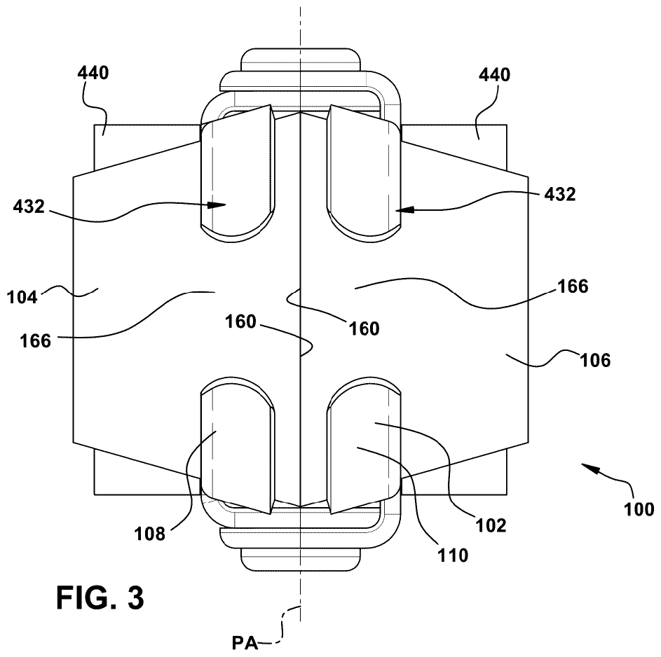

When the first and second wings 108, 110 are coupled together, selective manipulation of at least one of the actuating portions 440 moves the closure mechanism 102 selectively between opened (FIGS. 1-2) and closed (FIG. 3) conditions. In the closed condition, the actuating portions 440 are spaced from one another, while the engagement portions 432 are adjacent to one another. In the opened condition, the actuating portions 440 are adjacent and/or engage one another, while the engagement portions 432 are pivoted about the pivot axis PA away from one another.

The closure mechanism 102 may be urged to the closed condition by a biasing member 252. A user may apply a force to the actuating portions 440 to urge the actuating portions 440 toward one another against the urging of the biasing member 252 to move the closure mechanism 102 to the opened condition. As the actuating members 440 are urged toward one another, the engagement portions 432 are pivoted away from one another. Upon removal of the applied force, the biasing member 252 urges the actuating members 440 away from one another and, thus, urges the engagement portions 432 toward one another.

In the example configuration shown in FIG. 2, the biasing member 252 is a torsion spring formed from a nonmagnetic or paramagnetic material such as, but not limited to, titanium. In particular, the torsion spring biasing member 252 has a first spring end 254 contacting the base plate 412 of the first wing 108, a second spring end 256 contacting the base plate 412 of the second wing 110, and a spring body portion 258 coiled about the shaft 248 of the fastener 146 between first and second spring ends 254, 256. The biasing member 252, however, may be any other type of spring or biasing feature formed from any appropriate material and configured to bias the closure mechanism 102 into one of the opened and closed conditions through the use of spring force, resilient material(s), elastic deformation, one or more shape-memory materials, any other force or mechanical property, or any combination thereof.

Although the engagement portions 432 have been shown as being in the form of split rings 438, either or both of the engagement portions 432 may alternatively or additionally be/include any mechanism that provides a snap-fit engagement, a press-fit engagement, a magnetic engagement, an adhesive engagement, a frictional engagement, a screw and nut engagement, a hook and loop engagement, any other suitable engagement, or any combination thereof, between the closure mechanism 102 and the first and second rings 104, 106.

Each of the first and second rings 104, 106 may be any desired ring configured to selectively receive a portion of a vessel. In general, as shown in FIGS. 1-3, the first and second rings 104, 106 may each include oppositely facing first and second ring surfaces 160, 162, inner and outer surfaces 164, 166 extending transversely from the first ring surface 160 toward the second ring surface 162, and an inner lumen 168 defined by the inner surface 164. The inner surfaces 164 of the first and second rings 104, 106 may include tissue engagement features 270 for securing the first and second rings 104, 106 to a vessel. The tissue engagement features 270 may include, but are not limited to, one or more spikes, barbs, protrusions, textured areas, adhesives, or any combination thereof.

The engagement portions 432 of the first and second wings 108, 110 are each configured to selectively receive a respective one of the first and second rings 104, 106, though the device 100 may be configured such that the first and second rings 104, 106 are permanently fixed to their respective engagement portions 432. In particular, as is shown in FIGS. 1-3, each split ring 438 of the closure mechanism 102 contacts and extends around the outer surface 166 of a respective one of the first and second rings 104, 106. As shown in FIGS. 1-2, the first ring surfaces 160 of the first and second rings 104, 106 are configured to be spaced from one another when the closure mechanism 102 is in the opened condition. However, as shown in FIG. 3, the first ring surfaces 160 of the first and second rings 104, 106 are configured to be adjacent to and/or engage one another when the closure mechanism 102 is in the closed condition.

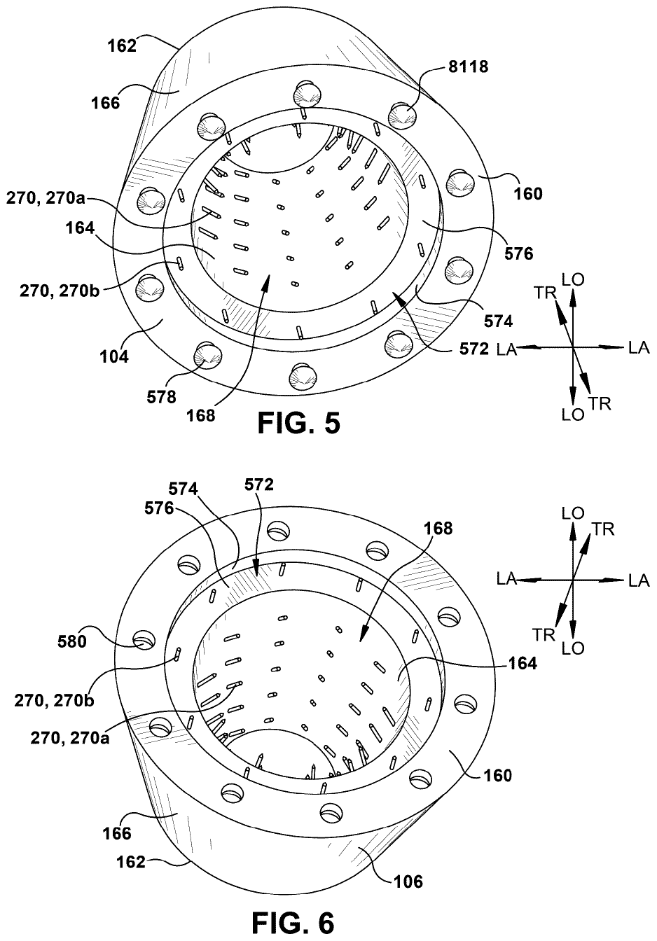

International Application No. PCT/US2021/035754 (hereafter referenced as “the '754 application”), filed 3 Jun. 2021 by Higdon et al. and tilted “AN APPARATUS FOR ANASTOMOSING A CUT VESSEL”, the subject matter of which is incorporated herein by reference in its entirety, discusses a variety of rings that are configured to selectively receive a portion of a vessel and that have the same general ring structure as described above. Therefore, each of the first and second rings 104, 106 may be, or may be a modified version of, any of the rings of the '754 application. As such, in the example configuration shown in FIG. 5, the first ring 104 has a diameter that inwardly tapers from a first ring surface 160 to a second ring surface 162. The first and second ring surfaces 160, 162 face in transversely opposite directions. The inward taper of the first ring 104 shapes the first ring 104 as a funnel, flute, or truncated cone. Inner and outer surfaces 164, 166 of the first ring 104 extend transversely from the first ring surface 160 toward the second ring surface 162. The first ring 104 has an inner lumen 168 that is defined by the inner surface 164. As shown in FIG. 5, corresponding to the shape of the first ring 104, the inner lumen 168 may be substantially shaped as a funnel, flute, or truncated cone.

The first ring 104 has an annular central recession 572 between the inner surface 164 and the first ring surface 160. The central recession 572 is defined by a recession sidewall 574 that extends substantially transversely from the first ring surface 160, and a recession floor 576 that extends substantially perpendicularly from the inner surface 164.

A plurality of tissue engagement features 270 in the form of spikes extend from at least one of the inner surface 164 and the recession floor 576 of the first ring 104. For example, a first set of spikes 270a may extend at about a 45-60 degree angle with respect to the inner surface 164 from the inner surface 164 into the inner lumen 168. A second set of spikes 270b may extend substantially perpendicularly from the recession floor 576. The second set of spikes 270b thus extend in the transverse direction. The first ring 104 may also include a plurality of posts 578 that extend substantially perpendicularly and transversely from the first ring surface 160.

As shown in FIG. 6, the structure of the second ring 106 substantially mirrors the structure of the first ring 104. Thus, the second ring includes oppositely facing first and second ring surfaces 160, 162, inner and outer surfaces 164, 166 extending transversely from the first ring surface 160 toward the second ring surface 162, an inner lumen 168, and a central recession 572 defined by a recession sidewall 574 and a recession floor 576. The second ring 106, and correspondingly the inner lumen 168 of the second ring 106, is also shaped as a funnel, flute, or truncated cone. The second ring 106 includes a first set of spikes 270a that may extend at about a 45-60 degree angle with respect to the inner surface 164 from the inner surface 164 into the inner lumen 168. The second ring 106 also includes a second set of spikes 270b that may extend substantially perpendicularly from the recession floor 576. However, unlike the first ring 104, the first ring surface 160 of the second ring 106 may include a plurality of receiver depressions 580 configured to receive the plurality of posts 578 when the first and second rings 104, 106 are joined together.

As an alternative, the first ring surface 160 of the second ring 106 may include at least one post 578, while the first ring surface 160 of the first ring 104 may include at least one receiver depression 580 configured to receive the at least one post 578. As another alternative, the first ring surface 160 of each of the first and second rings 104, 106 may include at least one post 578 and at least one receiver depression 580.

The first set of spikes 270a, the second set of spikes 270b, and/or the posts 578 can each be made out of the same materials as the first and second rings 104, 106. In such a configuration, the first set of spikes 270a, the second set of spikes 270b, and/or the posts 578 can each be integrally or monolithically formed as one-piece with the first and second rings 104, 106, or separately formed from and joined to the first and second rings 104, 106. Alternatively, the first set of spikes 270a, the second set of spikes 270b, and/or the posts 578 can each be made out of materials different from those used to form the first and second rings 104, 106. In such a configuration, the first set of spikes 270a, the second set of spikes 270b, and/or the posts 578 can each be separately formed from and joined to the first and second rings 104, 106. The first set of spikes first set of spikes 270a, the second set of spikes 270b, and/or the posts 578 can each be separately formed from and joined to the first and second rings 104, 106 by being bonded, sealed, adhered, press-fit, insert molded, overmolded, by any other suitable direct or indirect attachment or coupling means, or by any combination thereof.

It is contemplated that instead of including a plurality of posts 578 and receiver depressions 580 extending circumferentially on the first ring surfaces 160 of the first and second rings 104, 106, the first ring surfaces 160 may include any desired number of posts 578 and receiver depressions 580 only on the portions of the first ring surfaces 160 that are opposite the biasing member 252.

It is also contemplated that a ring locking mechanism can be provided on at least one of the first and second rings 104, 106 for selectively retaining the closure mechanism 102 in the closed condition. The ring locking mechanism may be a clip closure, a latch closure, a ratcheting member, an interlocking member, an adhesive, a hook and loop fastener, a magnetic fastener, a snap fastener, a hook and eye fastener, a string and eye fastener, any other suitable mechanism or technique for selectively retaining the closure mechanism 102 in the closed condition, or any combination thereof. The ring locking mechanism may be on one or more surfaces of at least one of the first and second rings 104, 106. The ring locking mechanism may be on at least one of the first and second rings 104, 106 opposite the biasing member 252. The posts 578 and the receiver depressions 580 can be omitted from the first and second rings 104, 106 if the ring locking mechanism is provided. One having ordinary skill in the art will be readily able to provide a suitable ring locking mechanism for a particular use environment.

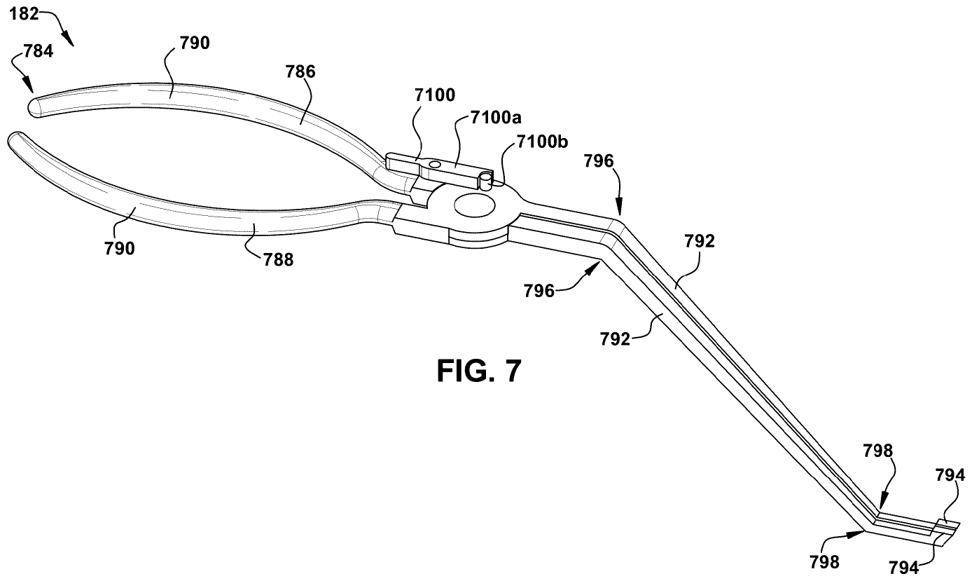

The anastomotic coupler device 100 as described above may be a part of a system 182 for anastomosing a cut vessel that also includes an applicator 784 (FIG. 7) for selectively engaging the actuating portions 440 of the closure mechanism 102 and selectively manipulating the actuating portions 440 to move the closure mechanism 102 between the opened and closed conditions

As shown in FIG. 7, the applicator 784 includes first and second elongated members 786, 788 mutually connected to one another in such a manner that they are selectively pivotable relative to one another. The first elongated member 786 includes a handle 790, an arm 792 extending from the handle 790, and a jaw 794 at an end of the handle 790 for selectively engaging the actuating portion 440 of the first wing 108. The arm 792 may have a first bend 796 (e.g., a 45 degree bend) adjacent the handle 790 and a second bend 798 (e.g., a 45 degree bend) adjacent the jaw 794. The second elongated member 788 similarly includes a handle 790, an arm 792 extending from the handle 790, and a jaw 794 at an end of the handle 790 for selectively engaging the actuating portion 440 of the second wing 110. The arm of the second elongated member 788 may have a first bend 796 (e.g., a 45 degree bend) adjacent the handle 790 and a second bend 798 (e.g., a 45 degree bend) adjacent the jaw 794.

The handles 790 are selectively manipulable to move the closure mechanism 102 between the opened and closed conditions. For example, the jaws 794 may be engaged to the actuating portions 440 when the closure mechanism 102 is in the closed condition. The handles 790 may then be pivoted toward one another, which responsively pivots the jaws 794 toward one another. The pivoting jaws 794 urge the actuating portions 440 toward one another against the biasing member 252 to manipulate the closure mechanism 102 into the opened condition. If desired, the locking mechanism 7100 of the applicator 784 may then be actuated to lock the applicator 784 in a position in which the closure mechanism 102 is retained in the open condition. For example, the locking mechanism 7100 may include a latch arm 7100a rotatably on one of the first and second elongated members 786, 788 (shown here as being on the first elongated member 786) and a projection 7100b fixedly on one of the first and second elongated members 786, 788 (shown here as being on the second elongated member 788). The latch arm 7100a thus may be rotated into engagement with the projection 7100b to lock the applicator 784 in a position in which the closure mechanism 102 is retained in the open condition. When the user desires to manipulate the closure mechanism 102 back to the closed condition, the user may simply unlock the applicator 784 via the locking mechanism 7100 and then pivot the handles 790 away from one another. The pivoting handles 790 responsively pivots the jaws 794 away from one another and thereby permits the biasing member 252 to return the closure member 102 to the closed condition.

Although the applicator 784 has been described as acting via the jaws 794 pivoting toward one another as the handles 790 are pivoted toward one another, the applicator 784 may instead be configured so that the jaws 794 pivot toward one another as the handles 790 are pivoted away from one another. One of ordinary skill in the art may readily design the operational details of a suitable applicator for a particular use environment, following the teachings described and shown herein.

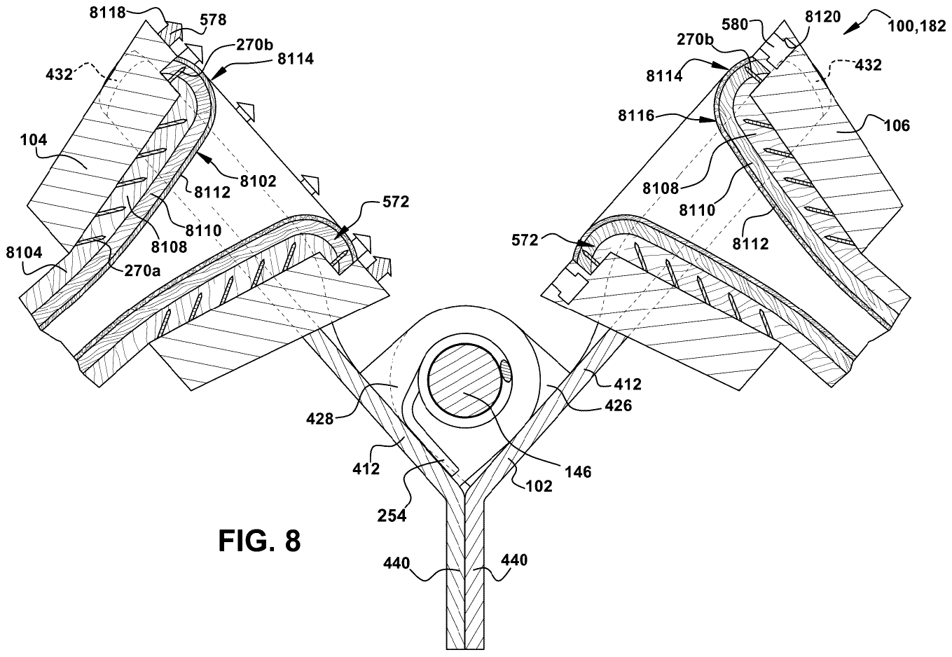

As shown in FIG. 8, in use, the closure mechanism 102 is manipulated into the opened condition against the urging of the biasing member 252. The closure mechanism 102 may be manipulated into the opened condition directly via a user or indirectly via the handles 790 of the applicator 784 in the manner described above. If provided, the locking mechanism 7100 can be actuated to lock the applicator 784 in a position in which the closure mechanism 102 is retained in the opened condition. The first ring 104 is then positioned over a first vessel end 8102 of a cut vessel 8104 of a patient so that the first vessel end 8102 engages the inner surface 164. The vessel 8104 can be any fluid carrying tube, such as, but not limited to, an artery, a vein, an arteriole, a capillary, a venule, and/or an intestine. The vessel 8104 is shown in FIG. 8 as an artery, for example. The first set of spikes 270a protrude into a vessel wall 8106 when the first vessel end 8102 engages the inner surface 164 to hold the first vessel end 8102 engaged to the first ring 104. An instrument, such as the rubber micro-plunger of the '754 application, can be used to gently ensure that the first set of spikes 270a penetrate into the vessel wall 8106. Because of their 45-60 degree extension, the first set of spikes 270a protrude into the outer layer (the adventitia) 8108 and the middle layer (the media) 8110 of the vessel wall 8106 without protruding into the inner most layer (the intima) 8112. The first set of spikes 270a thus protrude into the vessel wall 8106 without disrupting the inner lining (i.e., the inner most layer 8112) of the vessel 8104.

An edge 8114 of the first vessel end 8102 is then everted onto the central recession 572 of the first ring 104. The second set of spikes 270b penetrate into the vessel wall 8106 as the edge 8114 of the first vessel end 8102 is everted onto the central recession 572 to secure the first ring 104 to the edge 8114. The instrument/micro-plunger can be used to gently urge the second set of spikes 270b to penetrate into the vessel wall 8106. Similar, to the first set of spikes 270a, the second set of spikes 270a can be configured so that they penetrate the vessel wall 8106 without disrupting the inner lining (i.e., the inner most layer 8112) of the vessel 8104.

Before or after the first ring 104 is secured to the first vessel end 8102, but while the closure mechanism 102 is in the opened condition, the second ring 106 is secured to a second vessel end 8116 of the cut vessel 8104 in a similar manner as described above. It should be appreciated that the first and second rings 104, 106 of the anastomotic coupler device 100 shown in FIG. 8 have inner surfaces 164 that are inwardly tapered. The inward tapers of the inner surfaces 164 combined with the angular extensions of the first sets of spikes 270a with respect to the inwardly tapered inner surfaces 164 helps orient the first sets of spikes 270a into a desirable position for penetrating the first and second vessel ends 8102, 8116. Therefore, the inward tapers of the inner surfaces 164 combined with the angular extensions of the first sets of spikes 270a with respect to the inwardly tapered inner surfaces 164 help prevent the first and second vessel ends 8102, 8116 from undesirably egressing the first and second rings 104, 106.

Although the first and second rings 104, 106 have been described as being coupled to the closure mechanism 102 prior to being secured to their respective vessel ends 8102, 8116, the first and second rings 104, 106 may be secured to their respective vessel ends 8102, 8116 prior to being coupled to their respective engagement portions 432.

With the first and second rings 104, 106 secured to their respective vessel ends 8102, 8116, the applicator 784 (when provided) may be unlocked via the locking mechanism 7100. Once unlocked, the handles 790 may be manipulated in the manner described above to permit the biasing member 252 to return the closure mechanism 102 to the closed condition. The first and second rings 104, 106 are joined together so that the first surfaces 160 of the first and second rings 104, 106 abut one another as the biasing member 252 urges the closure mechanism 102 to the closed condition. The everted portions 8114 of the first and second vessel ends 8102, 8116 contact one another when the first and second rings 104, 106 are joined together. This end-to-end contact of the first and second vessel ends 8102, 8116 may lead to a successful anastomosis and help prevent the development of thrombosis.

The posts 578 of the first ring 104 may be received in the receiver depressions 580 of the second ring 106 as the first and second rings 104, 106 are joined together, when the posts 578 and the receiver depressions 580 are provided. This post-depression engagement helps to secure the first and second rings 104, 106 together and, thus, helps hold the closure mechanism 102 in the closed condition. At least one of the posts 578 and/or the receiver depressions 580 may have locking features (e.g., projections or barbs) to help prevent the posts 578 from undesirably egressing from the receiver depressions 580. For example, as shown in the example configuration of FIG. 8, the posts 578 can be substantially arrow-shaped and have at least one annular projection 8118 that is configured to selectively contact a complementary surface 8120 in the receiver depressions 580 to selectively prevent the posts 578 from undesirably egressing from the receiver depressions 580. However, the urging provided by the biasing member 252 could be adapted to, on its own, hold the first and second rings 104, 106 together and prevent the closure mechanism 102 from undesirably moving to the opened condition. In such a case, the posts 578 and the receiver depressions 580 may be omitted from the first and second rings 104, 106.

The engagement between the posts 578 and the receiver depressions 580 also helps prevent one of the first and second vessel ends 8102, 8116 from rotating laterally and/or longitudinally relative to the other of the first and second vessel ends 8102, 8116 once the first and second rings 104, 106 are secured together. If the anastomosed first and second vessel ends 8102, 8116 were to rotate relative to one another, at least one of the first vessel end 8102, the second vessel end 8116, and the vessel 8104 as a whole could become cinched or kinked, which may cause a delayed vessel thrombosis. The engagement between the posts 578 and the receiver depressions 580, however, prevents one of the first and second rings 104, 106 from rotating laterally and/or longitudinally relative to the other of the first and second rings 104, 106, which correspondingly helps prevent one of the first and second vessel ends 8102, 8116 from rotating laterally and/or longitudinally relative to the other of the first and second vessel ends 8102, 8116. Therefore, a medical professional can rotate each of the first and second vessel ends 8102, 8116 to a desired orientation, and then anastomose the first and second vessel ends 8102, 8116 by inserting the posts 578 into the receiver depressions 580 to prevent the first and second vessel ends 8102, 8116 from rotating laterally and/or longitudinally relative to one another.

After being manipulated to the closed condition, the user may re-manipulate the closure mechanism 102 to the opened condition for any reason, such as, for example, to inspect the first and second vessel ends 8102, 8116 within the first and second rings 104, 106, inspect the orientation of the first and second vessel ends 8102, 8116 or of the first and second rings 104, 106, or to assess spurt/backflow from the first and second vessel ends 8102, 8116. The closure mechanism 102 thus provides a relatively easy mechanism for moving the first and second rings 104, 106 (and, accordingly, the first and second vessel ends 8102, 8116) into and out of engagement with one another as desired.

The closure mechanism, the first ring 102, the second ring 104, the tissue engagement features 270 (e.g., the first set of spikes 270a and/or the second set of spikes 270b), the posts 578, and/or the applicator 784 can each be at least partially formed from silicone, rubber, polyvinyl chloride, polyethylene, polypropylene, nylon, stainless steel, titanium, any other metal, any other plastic, any other biocompatible material, or any combination thereof.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many use applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. The term “substantially” is used herein to indicate a quality that is largely, but not necessarily wholly, that which is specified—a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

I claim:

1. An apparatus for anastomosing a cut vessel, the apparatus comprising:
a first ring having an inner surface defining an inner lumen configured to selectively receive a first vessel end of the cut vessel;
a second ring having an inner surface that defines an inner lumen configured to selectively receive a second vessel end of the cut vessel; and
a closure mechanism having a first engagement portion joined to the first ring and a second engagement portion joined to the second ring, a biasing member of the closure mechanism being configured to urge the first and second rings toward one another to anastomose the cut vessel after the first and second vessel ends have been received in the first and second rings.

2. The apparatus of claim 1, wherein the closure mechanism has a closed condition in which the first and second rings are adjacent one another, and an opened condition in which the first and second rings are spaced from one another against the urging of the biasing member.

3. The apparatus of claim 2, wherein the closure mechanism has a first actuating portion connected to the first engagement portion and a second actuating portion connected to the second engagement portion, wherein selective manipulation of at least one of the first and second actuating portions moves the closure mechanism selectively between the closed and opened conditions.

4. A system for anastomosing a cut vessel, the system comprising:
the apparatus of claim 3;
an applicator for selectively engaging the first and second actuating portions of the closure mechanism and selectively manipulating the first and second actuating portions to move the closure mechanism between the opened and closed conditions.

5. The system of claim 4 wherein the applicator has a locking mechanism for selectively locking the applicator in a position in which the closure mechanism is retained in the opened condition.

6. The system of claim 4, wherein the applicator includes
a first handle, a first arm extending from the first handle, and a first jaw at an end of the first handle for selectively engaging the first actuating portion, and
a second handle, a second arm extending from the second handle, and a second jaw at an end of the second handle for selectively engaging the second actuating portion, the first and second handles being selectively manipulable to move the closure mechanism between the opened and closed conditions.

7. The system of claim 6, wherein
the first arm has a first bend adjacent the first handle and a second bend adjacent the first jaw, and
the second arm has a first bend adjacent the second handle and a second bend adjacent the second jaw.

8. The apparatus of claim 1, wherein the closure mechanism includes
a first wing having a first base plate, the first engagement portion connected to a first longitudinal end of the first base plate, and a first actuating portion connected to a second longitudinal end of the first base plate,
a second wing having a second base plate, the second engagement portion connected to a first longitudinal end of the second base plate, and a second actuating portion connected to a second longitudinal end of the second base plate, selective manipulation of at least one of the first and second actuating portions moving the closure mechanism selectively between closed and opened conditions.

9. The apparatus of claim 8, wherein
the first engagement portion includes first and second arcuate engagement arms that form a first split ring for selectively receiving the first ring, and
the second engagement portion includes first and second arcuate engagement arms that form a second split ring for selectively receiving the second ring.

10. The apparatus of claim 8, wherein
the first actuating portion includes a substantially flat first gripping surface connected to the second longitudinal end of the first base plate via a first bent portion of the first actuating portion, and
the second actuating portion includes a substantially flat second gripping surface connected to the second longitudinal end of the second base plate via a second bent portion of the second actuating portion.

11. The apparatus of claim 8, wherein
the first wing includes first and second connection projections extending from a transverse surface of the first base plate, each of the first and second connection projections of the first wing including an opening extending laterally therethrough, and
the second wing includes first and second connection projections extending from a transverse surface of the second base plate, each of the first and second connection projections of the second wing including an opening extending laterally therethrough, and
the closure mechanism includes a fastener that extends through each of the openings to pivotably join the first and second wings together.

12. The apparatus of claim 1, wherein the closure mechanism includes
a first wing having the first engagement portion and a first actuating portion, and
a second wing pivotably connected to the first wing, the second wing having the second engagement portion and a second actuating portion, selective manipulation of at least one of the first and second actuating portions pivoting at least one of the first and second wings relative to the other of the first and second wings.

13. The apparatus of claim 1, wherein
the inner surface of the first ring has at least one spike extending away from the inner surface into the inner lumen, the at least one spike of the first ring being configured to extend into a vessel wall of the first vessel end and thereby secure the first ring to the first vessel end, and
the inner surface of the second ring has at least one spike extending away from the inner surface into the inner lumen, the at least one spike of the second ring being configured to extend into a vessel wall of the second vessel end and secure the second ring to the second vessel end.

14. The apparatus of claim 13, wherein the at least one spike of the first ring extends at about a forty-five (45) to about a sixty (60) degree angle with respect to the inner surface of the first ring, and the at least one spike of the second ring extends at about a forty-five (45) to about a sixty (60) degree angle with respect to the inner surface of the second ring.

15. The apparatus of claim 1, wherein the first ring surface of the first ring includes at least one post configured to be received in a corresponding receiver depression on the first ring surface of the second ring when the first and second rings are joined together.

16. The apparatus of claim 15, wherein a portion of the at least one post is configured to engage a portion of the corresponding receiver depression to at least partially prevent the at least one post from egressing the corresponding receiver depression.

17. The apparatus of claim 1, wherein the biasing member is a spring.

18. A method anastomosing a cut vessel, the method comprising:
providing the apparatus of claim 1;
manipulating the closure mechanism into an opened condition;
with the closure mechanism in the opened condition, positioning the first ring over a first vessel end of the cut vessel so that the first vessel end is at least partially within the inner lumen and surrounded by the inner surface of the first ring;
with the closure mechanism in the opened condition, positioning the second ring over a second vessel end of the cut vessel so that the second vessel end is at least partially within the inner lumen and surrounded by the inner surface of the second ring; and
with the first and second rings positioned over the first and second vessels ends, manipulating the closure mechanism to a closed condition to urge the first and second rings toward one another and the first and second vessel ends into engagement with one another to anastomose the cut vessel.

19. The method of claim 18, wherein each of the first and second rings has at least one spike extending away from the inner surface into the inner lumen, the method further comprising:
penetrating a vessel wall of the first vessel end with the at least one spike of the first ring to secure the first ring to the first vessel end; and
penetrating a vessel wall of the second vessel end with the at least one spike of the second ring to secure the second ring to the second vessel end.

20. The method of claim 18, wherein
the first ring includes an annular central recession between the inner surface and a first ring surface, the annular recession being defined by a recession sidewall and a recession floor, the recession floor including at least one spike longitudinally extending away from the recession floor, and
the second ring includes an annular central recession between the inner surface and a second ring surface, the annular recession being defined by a recession sidewall and a recession floor, the recession floor including at least one spike longitudinally extending away from the recession floor, the second ring surface substantially facing the first ring surface when the closure mechanism is in the closed condition, the method further comprising:
everting an edge of the first vessel end onto the central recession of the first ring;
penetrating the vessel wall of the first vessel end with the at least one spike that extends away from the recession floor of the first ring to secure the first ring to the edge of the first vessel end;
everting an edge of the second vessel end onto the central recession of the second ring; and
penetrating the vessel wall of the second vessel end with the at least one spike that extends away from the recession floor of the second ring to secure the second ring to the edge of the second vessel end.
